(19) United States
(12) Patent Application Publication
(10) Pub. No.: US 2021/0186561 A1
(43) Pub. Date: Jun. 24, 2021

(54) BALLOON UNIT FOR UTERINE HEMOSTASIS

(71) Applicants: KYOTO UNIVERSITY, Kyoto-shi, Kyoto (JP); ATOM MEDICAL CORPORATION, Tokyo (JP)

(72) Inventors: Eiji Kondoh, Kyoto-shi, Kyoto (JP); Keisuke Wakabayashi, Saitama City, Saitama (JP); Ryoko Kakinuma, Saitama City, Saitama (JP); Shinichi Kobayashi, Saitama City, Saitama (JP); Tatsuhiko Seki, Saitama City, Saitama (JP); Ichiro Matsubara, Tokyo (JP)

(21) Appl. No.: 17/273,244

(22) PCT Filed: Sep. 5, 2019
(86) PCT No.: PCT/JP2019/034991

§ 371 Date: Mar. 3, 2021

(30) Foreign Application Priority Data
Sep. 5, 2018 (JP) 2018-166099

(51) Int. Cl. A61B 17/42; A61B 17/12; A61M 25/10

(52) U.S. Cl. CPC A61B 17/42; A61B 17/12136; A61B 17/12045; A61M 25/1011; A61M 2210/1433; A61M 25/10182; A61M 25/10186; A61B 2017/4216; A61B 2017/12004

(57) ABSTRACT

Provided is a balloon unit for uterine hemostasis capable of reliably suppressing or stopping uterine bleeding. The balloon unit includes a flexible tube and a balloon provided in a distal end portion of the tube. The balloon has a lower uterine lumen compression portion that compresses an inner surface of a lower uterine lumen and an upper uterine lumen compression portion that compresses an inner surface of an upper uterine lumen. As the balloon, two balloons including a first balloon functioning as the lower uterine lumen compression portion and a second balloon functioning as the upper uterine lumen compression portion are provided.

### TECHNICAL FIELD

The present invention relates to a balloon unit for uterine hemostasis that suppresses or stops uterine bleeding.

Priority is claimed on Japanese Patent Application No. 2018-166099, filed on Sep. 5, 2018, the content of which is incorporated herein by reference.

### BACKGROUND ART

In the related art, a balloon unit for hemostasis used for a Balloon Tamponade (BT method) is known as follows. A balloon for hemostasis is inserted from a vagina, and is disposed inside a uterus. Thereafter, a liquid is injected into the balloon to inflate the balloon. In this manner, uterine contraction is strengthened to suppress bleeding. The balloon unit configured in this way normally includes one balloon to be inserted into the uterus, a drain flow path for discharging blood or the like inside the uterus, and a supply-discharge flow path for injecting or discharging the liquid into or from the balloon. Normally, when the balloon is inserted into the uterus from an outside of body, the balloon is inserted into the vagina, and thereafter, the balloon is pushed into the uterus to be disposed in the uterus. Then, the one balloon is inflated by injecting the liquid into the balloon via a connector of the supply-discharge flow path provided in an end portion on a side opposite to the balloon.

On the other hand, as a balloon unit for hemostasis provided with two balloons, for example, a balloon tube instrument disclosed in Patent Literature 1 is known. The balloon tube instrument disclosed in Patent Literature 1 includes a tube shaft including a passage which enables communication from one end side to the other end side, a first balloon provided in an intermediate portion of the tube shaft, a second balloon disposed adjacent to the first balloon, a fluid route for injecting and discharging a fluid into and from the first balloon and the second balloon, and a fluid injection port and a fluid discharge port which provided in both end portions of the fluid route. The first balloon is disposed inside the uterus, and the second balloon is disposed in a state of being inflated inside the vagina. In this manner, the first balloon inside the uterus can be disposed in a state where the first balloon is reliably brought into close contact with a bleeding site (site on a uterine os side inside the uterus) in a case of placenta previa.

### CITATION LIST

### Patent Literature

[Patent Literature 1] JP-A-2016-221247

### SUMMARY OF INVENTION

### Technical Problem

As described above, the balloon unit for uterine hemostasis in the related art promotes uterine contraction to stop bleeding. Even according to Patent Literature 1 described above, the balloon is only brought into close contact with the bleeding site in the case of the placenta previa. Therefore, according to a configuration disclosed in Patent Literature 1, when there is bleeding in a site other than the uterine os side inside the uterus, the bleeding inside the uterus cannot be stopped in some cases. This disadvantage similarly occurs even in a configuration having one balloon.

The present invention is made in view of the above-described circumstances, and an object thereof is to provide a balloon unit for uterine hemostasis capable of reliably suppressing or stopping uterine bleeding.

### Solution to Problem

As a result of diligent researches, the present inventors have found that a bleeding site in a uterus is directly compressed to stop bleeding. In this regard, according to Patent Literature 1, an object is to pinch a uterine cervix between two balloons so that the balloon is held in a uterine lumen. In this manner, it is conceivable that a bleeding point is compressed. However, the point which can be compressed is limited to only a portion on a uterine os side inside the uterus. Here, the present inventors have discovered the followings. A cavity (upper lumen) having a diameter smaller than that of a lower lumen on an inner side (upper side) of the lower lumen is present in the uterine lumen, and the bleeding point is also present in the upper lumen (hereinafter, referred to as an upper uterine lumen and a lower uterine lumen). The present inventors have noticed the followings. The balloon used for uterine hemostasis in the related art does not match a uterine shape, and can cope with bleeding in the lower uterine lumen to some extent, but cannot cope with bleeding in the upper uterine lumen at all. Then, the present inventors have found the followings. Inner surfaces of the lower uterine lumen and the upper uterine lumen can be compressed by forming the balloon in a shape matching the uterine shape. Even in a case where there is bleeding from any site of the uterus, the bleeding in the uterus can be reliably suppressed or stopped.

That is, the balloon unit for uterine hemostasis according to the present invention includes a flexible tube and a balloon provided in a distal end portion of the tube. The balloon has a lower uterine lumen compression portion configured to compress an inner surface of a lower uterine lumen and an upper uterine lumen compression portion configured to compress an inner surface of an upper uterine lumen.

In the present invention, the inner surface of the lower uterine lumen can be compressed by the lower uterine lumen compression portion of the balloon, and the inner surface of the upper uterine lumen can be compressed by the upper uterine lumen compression portion. Therefore, the whole uterine lumen can be compressed to reliably suppress or stop the uterine bleeding.

As a preferable aspect of the balloon unit for uterine hemostasis according to the present invention, the balloon may be one balloon. As a more preferable aspect of the balloon, two balloons including a first balloon forming the lower uterine lumen compression portion and a second balloon forming the upper uterine lumen compression portion may be provided.

In addition, when the two balloons are provided, the second balloon in an inflated state may be disposed on a distal end side from the first balloon.

Furthermore, the tube may have a first tube in which the first balloon is disposed, and a second tube in which the second balloon is disposed and which can be inserted into the first balloon.

In the above-described aspect, the first balloon and the second balloon are configured to be separate from each other. Accordingly, for example, after the first balloon is inflated to compress the inner surface of the lower uterine lumen, even when the second balloon is not held by a doctor before the second balloon is inflated, the second balloon can be held by the first balloon in an inflated state and the tube. Therefore, labor and time for the doctor to hold the second balloon can be saved, and the second balloon can be reliably inflated inside the upper uterine lumen.

In addition, the second balloon in the inflated state is disposed on the distal end side from the first balloon. In this manner, it is possible to obtain the balloon unit for uterine hemostasis that matches the uterine shape. Furthermore, the two balloons are separately disposed in the two tubes. In this manner, as the balloon unit for uterine hemostasis, operability can be improved.

As a preferable aspect of the balloon unit for uterine hemostasis according to the present invention, maximum capacity of the first balloon may be larger than maximum capacity of the second balloon.

In the above-described aspect, the inner surface of the lower uterine lumen can be reliably compressed by the first balloon having the maximum capacity larger than the maximum capacity of the second balloon. Therefore, when the second balloon is inflated, the second balloon can be supported by the first balloon in the inflated state. Accordingly, the inner surface of the upper uterine lumen can be reliably compressed by the second balloon.

As a preferable aspect of the balloon unit for uterine hemostasis according to the present invention, the maximum capacity of the first balloon may be 450 ml to 880 ml, the maximum capacity of the second balloon may be 250 ml to 400 ml, and a ratio between the maximum capacity of the first balloon and the maximum capacity of the second balloon may be 1.8 to 2.2:1.

In the above-described aspect, a diameter of the first balloon before the liquid is injected may be larger than a diameter of the second balloon before the liquid is injected.

In the above-described aspect, the diameter of the first balloon before the liquid is injected may be 35 mm to 45 mm, and the diameter of the second balloon before the liquid is injected may be 19 mm to 29 mm.

In the above-described aspect, a length of the first balloon before the liquid is injected may be shorter than a length of the second balloon before the liquid is injected.

In the above-described aspect, the length of the first balloon before the liquid is injected may be 75 mm to 95 mm, and the length of the second balloon before the liquid is injected may be 80 mm to 110 mm.

In the above-described aspect, the diameter of the first balloon when the liquid of 500 ml is injected may be larger than the diameter of the second balloon when the liquid of 300 ml is injected.

In the above-described aspect, the diameter of the first balloon may be 35 mm to 45 mm in a state where the liquid is not injected. The diameter of the first balloon may be 95 mm to 105 mm when the liquid of 500 ml is injected. The diameter of the second balloon may be 19 mm to 29 mm in a state where the liquid is not injected. The diameter of the second balloon may be 65 mm to 75 mm when the liquid of 300 ml is injected.

In the above-described aspect, the length of the first balloon when the liquid of 500 ml is injected may be shorter than the length of the second balloon when the liquid of 300 ml is injected.

In the above-described aspect, the length of the first balloon may be 75 mm to 95 mm in a state where the liquid is not injected. The length of the first balloon may be 90 mm to 110 mm when the liquid of 500 ml is injected. The length of the second balloon may be 80 mm to 110 mm in a state where the liquid is not injected. The length of the second balloon may be 100 mm to 130 mm when the liquid of 300 ml is injected.

In addition, in the above-described aspect, the first tube may have a first drain flow path and a first supply-discharge flow path which are formed in the first balloon. The second tube may have a second drain flow path and a second supply-discharge flow path which are formed in the second balloon.

In the above-described aspect, the drain flow path and the supply-discharge flow path are provided for each of the first balloon and the second balloon. Accordingly, a body fluid such as blood inside the uterus can be discharged outward from each of the balloons, and the liquid can be separately supplied and discharged to and from each of the balloons.

Furthermore, in the above-described aspect, the first drain flow path may have an accommodation portion configured to accommodate the second balloon in a deflated state. A portion other than the accommodation portion in the first drain flow path may have an inner diameter which enables at least the second tube to be accommodated. The second balloon may be configured to be slidable between an accommodation position accommodated in the accommodation portion and a protruding position protruding from the accommodation portion.

In the above-described aspect, the second balloon in a deflated state and the second tube can be accommodated in the first drain flow path. Accordingly, compared to a case where the first tube and the second tube are respectively inserted into the uterus in an independent state, insertion resistance can be reduced. In addition, in a state where the first balloon is inflated in the lower uterine lumen and the inner surface of the lower uterine lumen is compressed, the second balloon can be inflated after being slid to the protruding position. Accordingly, the inside of the upper uterine lumen can be reliably compressed to stop bleeding by the second balloon, and slipping of the second balloon with respect to the upper uterine lumen can be suppressed.

In addition, in the above-described aspect, the second balloon in the deflated state may be accommodated inside the first tube. The first drain flow path may have an inner diameter through which the second balloon in the deflated state and the second tube can be inserted.

In the above-described aspect, the second balloon in the deflated state can be inserted and slid into the first drain flow path, and can be guided to the upper uterine lumen.

In addition, in the above-described aspect, a stylet that holds a shape of the tube may be provided. When the stylet is accommodated inside the second drain flow path, the stylet may be located from a position where a distal end of the stylet does not exceed a distal end of the second balloon to a proximal end portion of the second balloon.

Here, when the distal end of the stylet exceeds the distal end of the second balloon, there is a possibility that the stylet may protrude from the distal end of the second drain flow path. On the other hand, when the distal end of the stylet does not extend to the proximal end portion of the second balloon, the second balloon cannot be supported by the stylet. Accordingly, the second balloon is less likely to be inserted into the upper uterine lumen.

In contrast, in the above-described aspect, the distal end of the stylet is located from the position where the distal end of the stylet does not exceed the distal end of the second balloon to the proximal end portion of the second balloon. Accordingly while undesirable protruding of the stylet is suppressed, the balloon unit for uterine hemostasis can be properly inserted.

In addition, in the above-described aspect, the stylet may be formed of pure aluminum.

In the above-described aspect, the stylet is formed of the pure aluminum. Accordingly, the stylet can maintain a shape thereof at a bent angle, and can deform when a strong force is applied thereto. Therefore, operability of the balloon unit for uterine hemostasis can be improved.

In addition, in the above-described aspect, at least one of the first balloon and the second balloon may be provided with a detection member that can be detected by ultrasonic echo.

In the above-described aspect, at least one of the first balloon and the second balloon is provided with the detection member that can be detected by the ultrasonic echo. Accordingly, positions of the first balloon and the second balloon inside the uterus can be easily detected by the ultrasonic echo.

### Advantageous Effects of Invention

According to the present invention, the bleeding inside the uterus can be reliably suppressed or stopped.

### BRIEF DESCRIPTION OF DRAWINGS

FIG. 1 is a plan view of a balloon unit for uterine hemostasis according to an embodiment of the present invention.

FIG. 2 is a plan view when a second balloon in the above-described embodiment is viewed from a distal end side.

FIG. 3 is a cross-sectional view taken along arrow line A1-A1 illustrated in FIG. 1 of the second balloon in the above-described embodiment.

FIG. 4 is a cross-sectional view taken along arrow line B1-B1 illustrated in FIG. 1 of a first tube in which the second tube in the above-described embodiment is accommodated.

FIG. 5 is a longitudinal sectional view of a first balloon in which the second balloon according to the above-described embodiment is accommodated.

FIG. 6 is a perspective view of the balloon unit for uterine hemostasis in a state where the second balloon in the above-described embodiment is accommodated in the first balloon.

FIG. 7 is a perspective view illustrating the balloon unit for uterine hemostasis in a state where the first balloon and the second balloon in the above-described embodiment are inflated.

FIG. 8 is a side view of the balloon unit for uterine hemostasis in a state where the first balloon and the second balloon in the above-described embodiment are inflated.

FIG. 9 is a schematic side view illustrating a state where the balloon unit for uterine hemostasis in the above-described embodiment is disposed inside a uterus to compress an inner surface of a lower uterine lumen and an inner surface of an upper uterine lumen.

FIG. 10 is a schematic front view illustrating a state where the balloon unit for uterine hemostasis in the above-described embodiment is disposed inside the uterus to compress the inner surface of the lower uterine lumen and the inner surface of the upper uterine lumen.

### DESCRIPTION OF EMBODIMENTS

Hereinafter, an embodiment of a balloon unit for uterine hemostasis according to the present invention will be described with reference to the drawings.

### [Schematic Configuration of Balloon Unit for Uterine Hemostasis]

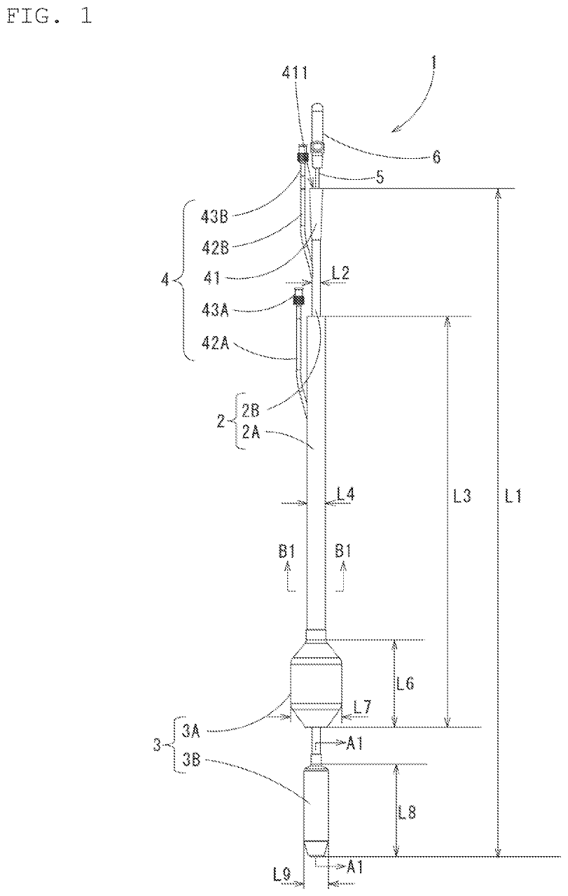

As illustrated in FIG. 1, a balloon unit for uterine hemostasis (hereinafter, referred to as a balloon unit) 1 according to the present embodiment includes a flexible tube 2, a balloon 3 provided in a distal end portion of the tube 2, and a connector 4 provided in the tube 2.

In the balloon unit 1, a side on which the balloon 3 is provided will be referred to as a distal end side, and a side opposite thereto will be referred to as a proximal end side.

### [Configuration of Tube]

The tube 2 has a first tube 2A and a second tube 2B. For example, each of the tubes 2A and 2B is formed of a synthetic resin such as polyvinyl chloride, silicone rubber, and a thermoplastic elastomer, and is flexible. A line (not illustrated) is formed in each of the tubes 2A and 2B along an extending direction of each of the tubes 2A and 2B. The line is formed in such a way that a material which can be detected by an X-ray is applied to or mixed with each of the tubes 2A and 2B. Out of these, the second tube 2B is formed to have a length approximately twice (1.8 to 2.2 times) that of the first tube 2A. Specifically, a length L3 of the first tube 2A is set to 360 mm to 400 mm, a length L1 of the second tube 2B is set to 660 mm to 880 mm, and a ratio between L3 and L1 is L3:L1=1:1.8 to 2.2. In addition, a diameter of the second tube 2B is formed to be smaller than a diameter of the first tube 2A. Each of the first tube 2A and the second tube 2B will be described in detail below.

### [Configuration of Second Tube]

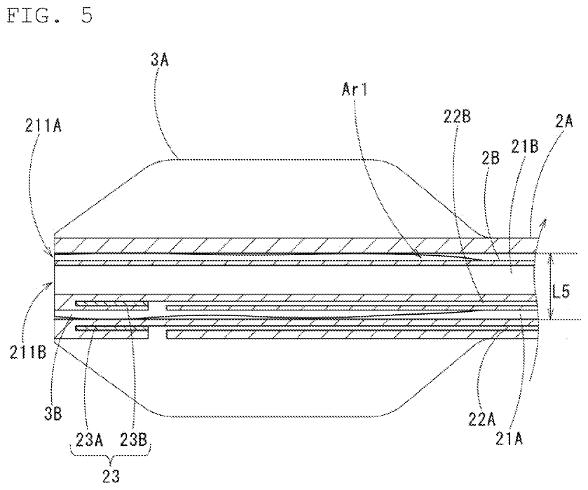

As illustrated in FIG. 1, a second balloon 3B is provided in a distal end portion of the second tube 2B, and a drain terminal 41 is connected to a proximal end portion of the second tube 2B. The length L1 of the second tube 2B (length from the distal end of the second tube 2B to the proximal end portion of the drain terminal 41) is set to 660 mm to 880 mm. The first tube 2A has a first drain flow path 21A which is open on the distal end side of the first balloon 3A to discharge the blood inside the uterus to the outside. As illustrated in FIG. 4, the second tube 2B is set to have an outer diameter which is approximately half (0.4 to 0.6 times) of an inner diameter L5 (16 mm to 20 mm) of the first drain flow path 21A. Specifically, an outer diameter L2 of the second tube 2B is set to 6.4 mm to 12 mm. In other words, a ratio between L5 and L2 is L5:L2=1:0.4 to 0.6. Therefore, as illustrated in FIG. 4, even in a state where the second tube 2B is inserted into the first drain flow path 21A, a sufficient gap is formed to allow a body fluid to flow between the first tube 2A and the second tube 2B.

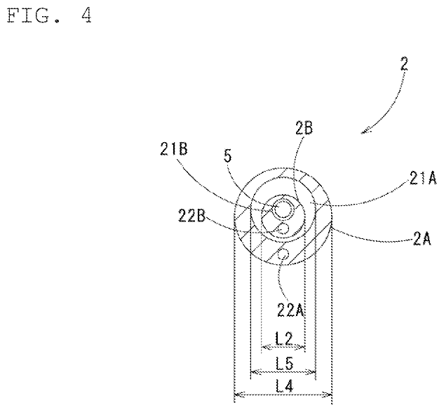

In addition, as illustrated in FIG. 3, the second tube 2B includes a second drain flow path 21B which is provided to penetrate the second balloon 3B and is open outward of the second balloon 3B on the distal end side of the second balloon 3B to discharge the blood inside the uterus to the outside, and a second supply-discharge flow path 22B communicating with the second balloon 3B. The inner diameter of the second drain flow path 21B is set to 4.2 mm to 5.2 mm, and the inner diameter of the second supply-discharge flow path 22B is set to 1.1 mm to 2.6 mm. Out of these, the second drain flow path 21B communicates with a portion from the distal end to the proximal end of the second tube 2B. As illustrated in FIG. 3, a distal end portion thereof has an opening 211B into which the blood inside the uterus flows. On the other hand, the second supply-discharge flow path 22B communicates with a portion from the vicinity of the distal end to the vicinity of the proximal end of the second tube 2B. That is, the second supply-discharge flow path 22B does not reach either the distal end or the proximal end of the second tube 2B. A second opening 222B is provided inside the second balloon 3B of the second supply-discharge flow path 22B.

Here, the second tube 2B is provided with the line formed in such a way that the material which can be detected by the X-ray is applied thereto or mixed therewith as described above. Accordingly, although the position of the second tube 2B can be detected by the X-ray, when there is no X-ray inspection device, the position cannot be detected even when the second tube 2B of the balloon unit 1 is inserted into the uterus. In order to cope with this situation, in the present embodiment, as illustrated in FIG. 3, a detection member 23B which can be detected by the ultrasonic echo is sealed on the distal end side of the second supply-discharge flow path 22B in the second tube 2B. The detection member 23B is formed of a metal wire rod which can be detected by the ultrasonic echo. Therefore, even when there is no X-ray inspection device, the position of the second balloon 3B can be easily detected by the ultrasonic echo.

The detection member 23 (detection member 23B and detection member 23A (to be described later)) is formed of the metal which can be detected by the ultrasonic echo. However, preferably, the detection member 23 is formed of a non-ferrous metal or stainless steel which is resistant to rust in the metal. More preferably, the detection member 23 is formed of aluminum which less affects a living body. In this case, the detection member 23 may be formed of pure aluminum having the same composition as that of a stylet 5 (to be described later).

### [Configuration of First Tube]

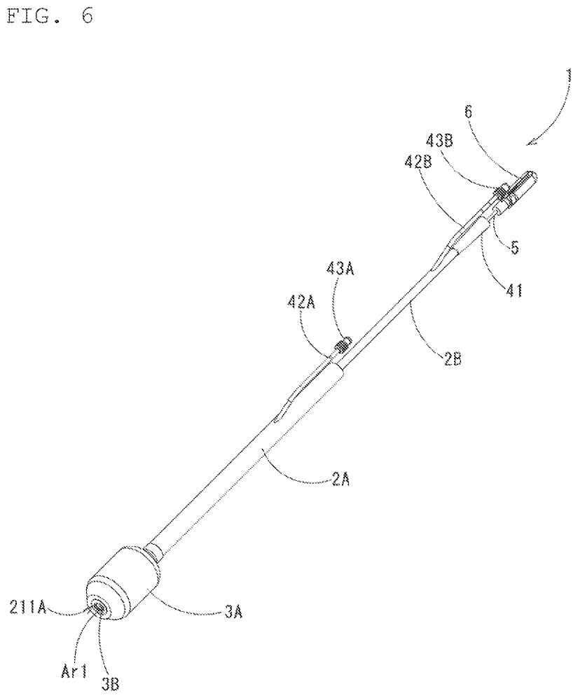

As illustrated in FIG. 1, the first balloon 3A is provided in the distal end portion of the first tube 2A. The length L3 of the first tube 2A is set to 360 mm to 400 mm, and the outer diameter L4 is set to 20 mm to 26 mm. As illustrated in FIG. 5, the first tube 2A includes a first drain flow path 21A which penetrates the first balloon 3A and is open outward of the first balloon 3A on the distal end side of the first balloon 3A to discharge the blood inside the uterus to the outside, and a first supply-discharge flow path 22A communicating with the first balloon 3A. The inner diameter L5 of the first drain flow path 22A is set to 16 mm to 20 mm, and the inner diameter of the first supply-discharge flow path 22A is set to 1.1 mm to 2.6 mm. Out of these, the first drain flow path 21A communicates with a portion from the distal end to the proximal end of the first tube 2A. As illustrated in FIG. 5, the distal end portion has an opening 211A through which the blood inside the uterus flows. On the other hand, the first supply-discharge flow path 22A communicates with a portion from the vicinity of the distal end to the vicinity of the proximal end of the first tube 2A. That is, the first supply-discharge flow path 22A does not reach either the distal end or the proximal end of the first tube 2A. A first opening 222A is provided inside the first balloon 3A of the first supply-discharge flow path 22A.

As illustrated in FIG. 5, the first drain flow path 21A of the first tube 2A has an accommodation portion Ar1 that accommodates the second balloon 3B in a deflated state. As illustrated in FIG. 4, the inner diameter L5 of a portion other than the accommodation portion Ar1 is formed to be a dimension which enables at least the second tube 2B to be accommodated. Specifically, the inner diameter L5 (16 mm to 20 mm) of the first drain flow path 21A is set to be approximately twice the outer diameter L2 (6.4 mm to 12 mm) of the second tube 2B. In other words, a ratio between L5 and L2 is L5:L2=1:0.4 to 0.6. In the present embodiment, the first drain flow path 21A has a shape extending straight, and the inner diameter L5 is set so that both the second balloon 3B in a deflated state and the second tube 2B can be inserted. Therefore, the second balloon 3B is slidable between an accommodation position accommodated in the accommodation portion Ar1 and a protruding position (position illustrated in FIG. 1) protruding from the accommodation portion Ar1.

In the present embodiment, the first drain flow path 21A has the shape extending straight. However, the configuration is not limited thereto. For example, the diameter of the portion serving as the accommodation portion Ar1 may be set to be larger than the inner diameter L5 of the portion other than the accommodation portion Ar1.

In addition, the detection member 23A similar to the above-described detection member 23B is sealed on the distal end side of the first supply-discharge flow path 22A. Therefore, even when there is no X-ray inspection device, the position of the first balloon 3A can be easily detected by the ultrasonic echo.

### [Configuration of Balloon]

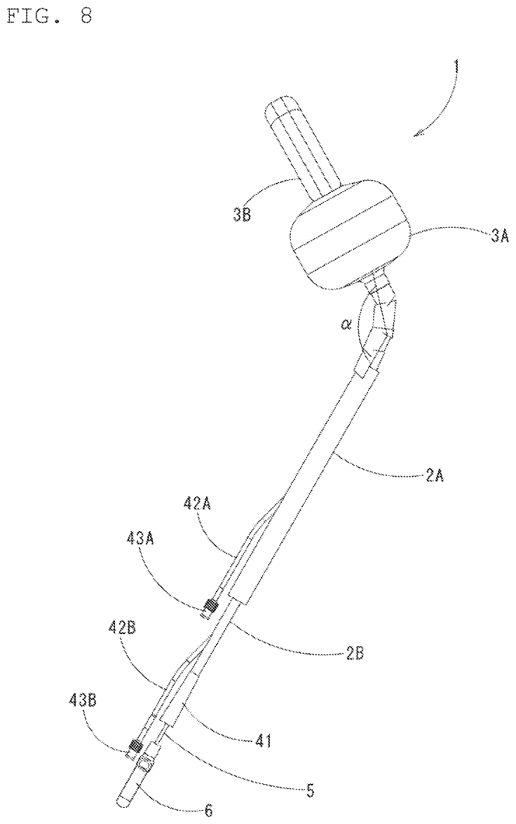

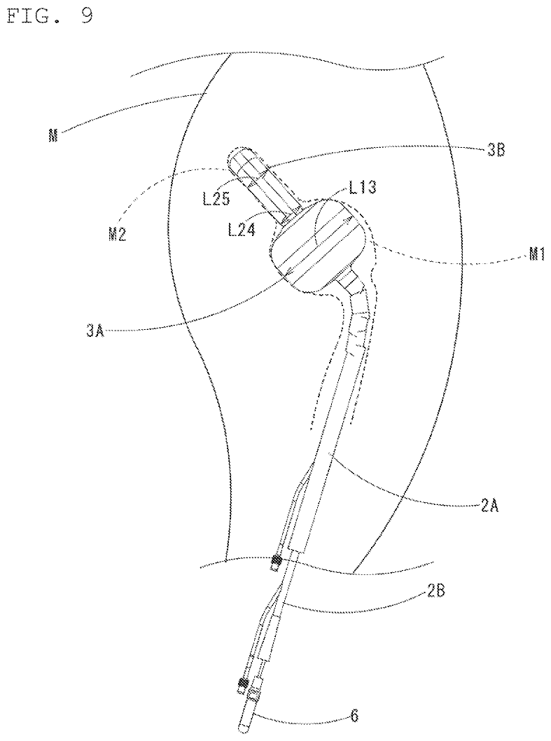

For example, the balloon 3 is formed of silicone rubber or the like. The balloon 3 has a first balloon 3A communicating with the first supply-discharge flow path 22A and a second balloon 3B communicating with the second supply-discharge flow path 22B, and is inflated by the liquid such as water injected via each of the supply-discharge flow paths 22A and 22B. As illustrated in FIGS. 7 and 8, in an inflated state, the first balloon 3A is formed so that the length is shorter and the outer diameter is larger than those of the second balloon 3B. In other words, in the inflated state, the second balloon 3B is formed so that length is longer and the outer diameter is smaller than those of the first balloon 3A.

Out of these, the first balloon 3A is configured so that the liquid having a volume approximately twice (1.8 to 2.2 times) that of the second balloon 3B can be injected. For example, the liquid of maximum 450 ml to 880 ml can be injected. On the other hand, the second balloon 3B is configured so that the liquid of maximum 250 ml to 400 ml can be injected. A ratio between maximum capacity of the first balloon 3A and maximum capacity of the second balloon 3B is the maximum capacity of the first balloon 3A: the maximum capacity of the second balloon 3B=1.8 to 2.2:1. The maximum capacity of the liquid can be injected into each of the balloons 3A and 3B. However, when the balloon is inserted into the uterus, a proper amount according to a uterine shape is injected. More specifically, a proper amount according to a shape of a lower uterine lumen M1 is injected into the first balloon 3A, and a proper amount according to a shape of an upper uterine lumen M2 is injected into the second balloon 3B.

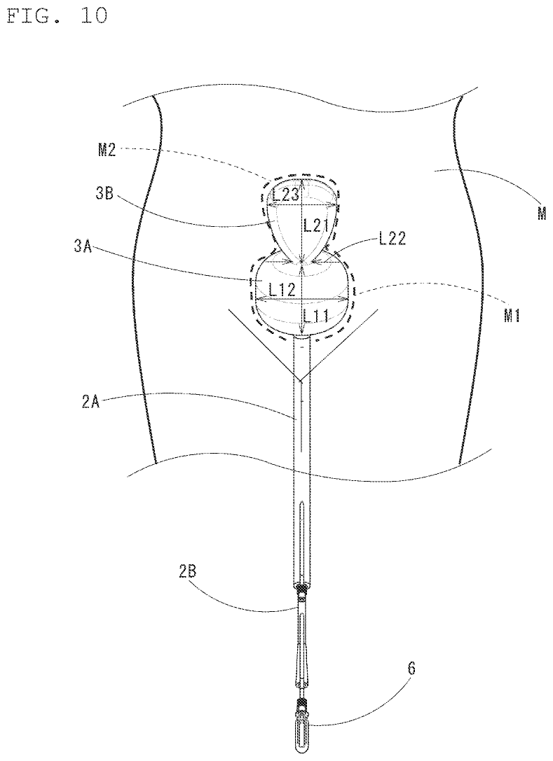

In addition, a length L6 of the first balloon 3A in a state where the liquid is not injected is set to 75 mm to 95 mm, and a diameter L7 is set to 35 mm to 45 mm. In contrast, the length L6 of the first balloon 3A when the liquid of 500 ml is injected is set to 90 mm to 110 mm, and the diameter L7 is set to 95 mm to 105 mm. When the liquid is injected into the first balloon 3A in this way, the first balloon 3A has a substantially spherical shape as illustrated in FIGS. 7 and 8. The first balloon 3A is inflated inside the uterus to compress the inner surface of the lower uterine lumen M1 as illustrated in FIGS. 9 and 10. That is, the first balloon 3A functions as a lower uterine lumen compression portion according to the present invention.

On the other hand, the second balloon 3B is configured so that the liquid having approximately half the volume of the first balloon 3A can be injected. As described above, the liquid of maximum 250 ml to 400 ml can be injected. Specifically, a length L8 of the second balloon 3B is 80 mm to 110 mm, and the diameter L9 is 19 mm to 29 mm in a state where the liquid is not injected. In contrast, the length L8 of the second balloon 3B is 100 mm to 130 mm, and the diameter L9 is 65 mm to 75 mm when the liquid of 300 ml is injected. When the liquid is injected into the second balloon 3B in this way, the second balloon 3B has a substantially columnar shape as illustrated in FIGS. 7 and 8. However, when accommodated in the upper uterine lumen M2, as illustrated in FIGS. 9 and 10, the second balloon 3B has a flat shape according to a shape thereof. As illustrated in FIGS. 9 and 10, the second balloon 3B is inflated inside the uterus to be located on an inner side (upper side) from the lower uterine lumen M1, and functions as an upper uterine lumen compression portion that compresses the inner surface of the upper uterine lumen M2 having the diameter smaller than that of the lower uterine lumen M1. A size (maximum capacity and various diameters) of each of the balloons 3A and 3B can be set in any desired way.

When the liquid is injected into each of the first balloon 3A and the second balloon 3B, a gap is not formed between the first balloon 3A and the second balloon 3B as illustrated in FIGS. 7 and 8. In this manner, the second balloon 3B is prevented from slipping out of the upper uterine lumen M2. However, in order to facilitate understanding, FIG. 1 illustrates a state where the second balloon 3B is separated from the distal end of the first balloon 3A.

### [Configuration of Connector]

The connector 4 includes the drain terminal 41 communicating with the second drain flow path 21B and provided in the proximal end portion of the second tube 2B, the flexible second supply-discharge tube 42B communicating with the second supply-discharge flow path 22B on the distal end side from the drain terminal 41 and extending by branching outward of the second tube 2B, the second supply-discharge terminal 43B provided in the proximal end of the second supply-discharge tube 42B and communicating with the second supply-discharge tube 42B, the flexible first supply-discharge tube 42A communicating with the first supply-discharge flow path 22A and extending by branching outward of the first tube 2A, and the first supply-discharge terminal 43A provided in the proximal end of the first supply-discharge tube 42A and communicating with the first supply-discharge tube 42A.

The drain terminal 41 is a flexible tubular member formed of silicone rubber. The diameter of the drain terminal 41 is gradually enlarged toward the proximal end side. The drain terminal 41 communicates with the second drain flow path 21B. Accordingly, the blood inside the uterus which flows from the opening 211B is discharged from an opening 411 (refer to FIG. 1) of the drain terminal 41 via the second drain flow path 21B. A cap 6 connected to the stylet 5 is mounted on the opening 411 of the drain terminal 41.

For example, the respective supply-discharge tubes 42A and 42B are formed of silicone rubber or the like, and are flexible. The distal ends of the respective supply-discharge tubes 42A and 42B communicate with the supply-discharge flow paths 22A and 22B inside the respective tubes 2A and 2B, and extend outward from the vicinity of the proximal end portions of the respective tubes 2A and 2B. The respective supply-discharge terminals 43A and 43B are provided in the proximal ends of the respective supply-discharge tubes 42A and 42B. For example, the respective supply-discharge terminals 43A and 43B are formed of hard polyvinyl chloride (PVC) or the like. A two-way cock (not illustrated) is connected to each of the supply-discharge terminals 43A and 43B.

A liquid dispenser such as a syringe (not illustrated) is connected to the above-described two-way cock. When the liquid is injected from the liquid dispenser via the two-way cock, the liquid is supplied into the respective balloons 3A and 3B via the respective supply-discharge terminals 43A and 43B, the respective supply-discharge tubes 42A and 42B, and the respective supply-discharge flow paths 22A and 22B. On the other hand, when the two-way cock is unlocked in a state where the liquid is supplied into the respective balloons 3A and 3B, the liquid inside the respective balloons 3A and 3B flows back to deflate the respective balloons 3A and 3B.

### [Configuration of Stylet and Cap]

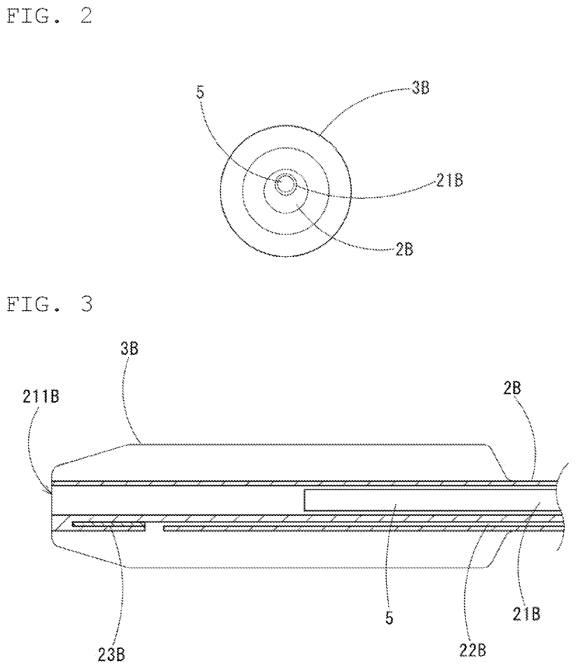

The stylet 5 is disposed inside the second drain flow path 21B as illustrated in FIGS. 2 and 4. The cap 6 detachably fitted to the opening 411 of the drain terminal 41 is fixed to the proximal end portion of the stylet 5. The cap 6 functions as a holder for putting a finger on the stylet 5 when operated by a medical worker. Out of these, the stylet 5 is formed of a wire of stainless steel, polypropylene, or the like, in addition to pure aluminum or an aluminum alloy (for example, A1070, A1080, or similar). In this manner, the stylet 5 can maintain a shape thereof at a bent angle, and deforms when a strong force is applied thereto.

Here, when the distal end of the stylet 5 exceeds the distal end of the second balloon 3B, there is a possibility that the stylet 5 may protrude from the distal end of the second drain flow path 21B. On the other hand, when the distal end of the stylet 5 does not extend to the proximal end portion of the second balloon 3B, the second balloon 3B cannot be supported by the stylet 5. Accordingly, the second balloon 3B is less likely to be inserted into the upper uterine lumen M2.

In the present embodiment, the length of the stylet 5 is set to the length in which the stylet 5 is located from a position where the distal end of the stylet 5 does not exceed the distal end of the second balloon 3B to the proximal end portion of the second balloon 3B, when the cap 6 is mounted on the opening 411 of the drain terminal 41. In this manner, the balloon unit 1 can be properly inserted into a mother's body while undesirable protruding of the stylet 5 is suppressed.

It is desirable that the length of the stylet 5 is set to such a dimension that the distal end of the stylet 5 is disposed at an intermediate position of the length of the second balloon 3B, in a state where the cap 6 is mounted on the opening 411 of the drain terminal 41 (refer to FIGS. 3 and 5).

The stylet 5 is formed to have the outer diameter so that a slight gap is formed between the stylet 5 and the second tube 2B in a state of being inserted into the second drain flow path 21B.

### [Using Method of Balloon Unit for Uterine Hemostasis]

FIGS. 9 and 10 are a schematic side view and a front view which illustrate a state where the balloon unit 1 is disposed inside the uterus to compress the lower uterine lumen M1 and the upper uterine lumen M2. In FIGS. 9 and 10, in order to facilitate understanding each configuration, there are vacant gaps between the first balloon 3A and the inner surface of the lower uterine lumen M1 and between the second balloon 3B and the inner surface of the upper uterine lumen M2. However, actually, the respective balloons 3A and 3B compress the above-described respective inner surfaces without any gap.

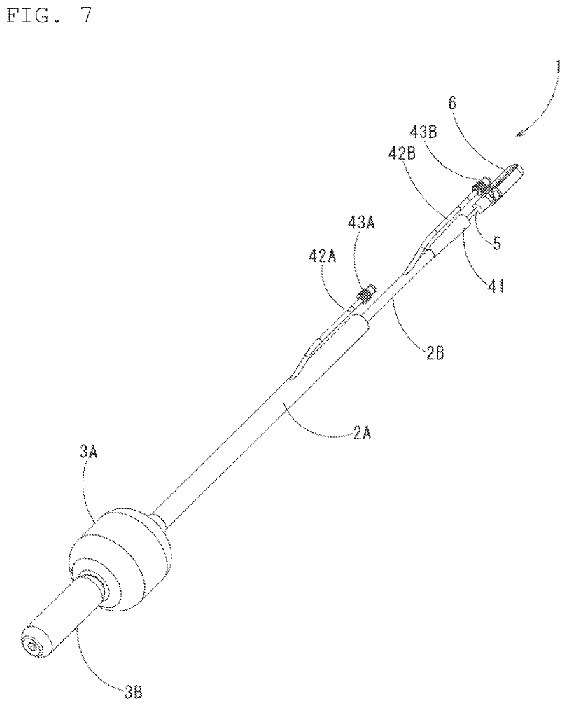

For example, the balloon unit 1 as described above is inserted into a mother's body M as illustrated in FIG. 9. First, the balloon unit 1 is gripped, and the tubes 2A and 2B are bent at a desired angle α (refer to FIG. 8). Thereafter, in a state where the second balloon 3B is accommodated in the accommodation portion Ar1 of the first tube 2A (state illustrated in FIG. 6), the first balloon 3A is inserted into the vagina, and is inserted into the uterus (lower uterine lumen M1) via the uterine os. The desired angle α is most preferably 135°. However, the angle may fall within a range of 120 to 150°, and can be appropriately changed according to the uterine shape of the mother's body M.

Then, the liquid flows only into the first balloon 3A from the first opening 222A via the first supply-discharge tube 42A and the first supply-discharge flow path 22A. In this manner, the inner surface of the lower uterine lumen M1 is in a state of being compressed by the first balloon 3A. Specifically, as various dimensions when the liquid is injected into the first balloon 3A inside the lower uterine lumen M1, a vertical dimension L11 (refer to FIG. 10) when the first balloon 3A is viewed from the front surface is 50 mm to 100 mm. A horizontal dimension L12 is 50 mm to 90 mm. A horizontal dimension L13 (refer to FIG. 9) is 40 mm to 100 mm when the first balloon 3A is viewed from the side surface. Accordingly, the inner surface of the lower uterine lumen M1 can be compressed by the first balloon 3A.

The inner surface of the lower uterine lumen M1 is compressed by the first balloon 3A. Thereafter, the cap 6 connected to the stylet 5 is gripped, and is pushed inward. In this manner, the second tube 2B and the second balloon 3B slide inside the first drain flow path 21A of the first tube 2A, and the second balloon 3B protrudes to the inner side (distal end side) from the first balloon 3A. Then, the second balloon 3B reaches the upper uterine lumen M2. Thereafter, the liquid flows into the second balloon 3B from the second opening 222B via the second supply-discharge tube 42B and the second supply-discharge flow path 22B. In this manner, the inner surface of the upper uterine lumen M2 is in a state of being compressed by the second balloon 3B (state illustrated in FIGS. 9 and 10).

Specifically, when the liquid is injected into the second balloon 3B, as illustrated in FIGS. 9 and 10, the second balloon 3B has a flat shape according to the shape of the upper uterine lumen M2. More specifically, a vertical dimension L21 when the second balloon 3B is viewed from the front surface is 70 mm to 140 mm. A horizontal dimension L22 of an end portion on the first balloon 3A side is 15 mm to 45 mm. A horizontal dimension L23 of a maximum width portion on the distal end side of the second balloon 3B is 45 mm to 90 mm. A horizontal dimension L24 of an end portion on the first balloon 3A side when the second balloon 3B is viewed from the side surface is 5 mm to 40 mm. A horizontal dimension L25 of a maximum width portion on the above-described distal end side is 5 mm to 40 mm. Accordingly, the inner surface of the upper uterine lumen M2 can be compressed by the second balloon 3B.

In this way, the inner surfaces of the lower uterine lumen M1 and the upper uterine lumen M2 are respectively compressed by the first balloon 3A and the second balloon 3B. Accordingly, even when there is bleeding at any position on the inner surfaces in the lower uterine lumen M1 and the upper uterine lumen M2, the bleeding can be reliably suppressed or stopped. In addition, the body fluid such as the blood can be discharged from each of the lower uterine lumen M1 and the upper uterine lumen M2 via the opening 211A, the opening 211B, and the respective drain flow paths 21A and 21B.

In this case, even when the second tube 2B is disposed in an inserted state, the first drain flow path 21A has a sufficient gap. Accordingly, the body fluid is discharged through the gap. In the second drain flow path 21B, the second balloon 3B is guided to the upper uterine lumen M2. Thereafter, the body fluid can be discharged from the second drain flow path 21B by pulling out the stylet 5 from the second drain flow path 21B.

In the present embodiment, the stylet 5 is formed of the pure aluminum. Accordingly, the stylet 5 can maintain a shape thereof at a bent angle, and deforms when a strong force is applied thereto. Therefore, operability of the balloon unit 1 can be improved. Therefore, an angle at which the respective balloons 3A and 3B can be easily inserted into the lower uterine lumen M1 and the upper uterine lumen M2 (angle α of the first tube 2A illustrated in FIG. 8) can be appropriately set.

### [Other Using Method]

In the above-described using method, an example has been described in which the balloon unit 1 is used in a state illustrated in FIG. 6, that is, in a state where the second balloon 3B and the second tube 2B are accommodated in the first drain flow path 21A of the first tube 2A. However, the configuration is not limited thereto. For example, a unit including the first tube 2A and the first balloon 3A and a unit including the second tube 2B and the second balloon 3B may be used separately from each other. In this case, only when the bleeding inside the uterus cannot be suppressed by disposing and inflating the first balloon 3A inside the lower uterine lumen M1 to compress the inner surface, the second balloon 3B and the second tube 2B may be inserted into the first drain flow path 21A of the first tube 2A, and the second balloon 3B is disposed and inflated inside the upper uterine lumen M2 to compress the inner surface of the upper uterine lumen M2. That is, when the bleeding can be suppressed only by the first tube 2A and the first balloon 3A, it is not necessary to use the unit including the second tube 2B and the second balloon 3B. Accordingly, the second tube 2B and the second balloon 3B do not need to be contaminated. Therefore, the using method is economically excellent.

In addition, in the above-described embodiment, a method of inserting the balloon unit 1 into the uterus of the mother's body M by a normal method (method of inserting the balloon unit 1 from the vagina) has been described. However, the present invention is also properly applicable to a method of inserting the balloon unit 1 through an incised portion after a Caesarean section is performed.

In the present embodiment, the inner surface of the lower uterine lumen M1 can be compressed by the first balloon 3A that functions as the lower uterine lumen compression portion of the balloon 3, and the inner surface of the upper uterine lumen M2 can be compressed by the second balloon 3B that functions as the upper uterine lumen compression portion. Accordingly, the bleeding inside the uterus can be reliably suppressed or stopped. In addition, the first balloon 3A and the second balloon 3B are configured to be separate from each other, and the supply-discharge flow paths 22A and 22B are distributed in each of the first balloon 3A and the second balloon 3B. Accordingly, after the first balloon 3A is inflated to compress the inner surface of the lower uterine lumen M1, even when the second balloon 3B is not held by a doctor before the second balloon 3B is inflated, the second balloon 3B can be held by the first balloon 3A in an inflated state. Therefore, labor and time for the doctor to hold the second balloon 3B can be saved, and the second balloon 3B can be reliably inflated inside the upper uterine lumen M2. Furthermore, the maximum capacity of the first balloon 3A (for example, 450 ml to 880 ml) is larger than the maximum capacity of the second balloon 3B (for example, 250 ml to 400 ml), and a ratio between the maximum capacity of the first balloon 3A and the maximum capacity of the second balloon 3B is 1.8 to 2.2:1. Accordingly, the inner surface of the lower uterine lumen M1 can be reliably compressed by the first balloon 3A. When the second balloon 3B is inflated, the second balloon 3B can be supported by the first balloon 3A in an inflated state. Therefore, the inner surface of the upper uterine lumen M2 can be reliably compressed by the second balloon 3B.

In the present embodiment, the second balloon 3B in a deflated state and the second tube 2B can be accommodated inside the first drain flow path 21A. Accordingly, compared to a case where the first tube 2A and the second tube 2B are respectively inserted into the uterus in an independent state, insertion resistance into the uterus can be reduced. In addition, in a state where the first balloon 3A is inflated in the lower uterine lumen M1 to compress the inner surface of the lower uterine lumen M1, the second balloon 3B can be slid to and inflated at the protruding position. Accordingly, the inner surface of the upper uterine lumen M2 can be reliably compressed by the second balloon 3B to stop the bleeding.

The present invention is not limited to the above-described embodiment, and various modifications can be added within the scope not departing from the concept of the present invention. For example, in the above-described embodiment, the balloon 3 has each of the first balloon 3A and the second balloon 3B. However, the configuration is not limited thereto. That is, the first balloon 3A and the second balloon 3B may be integrated with each other. In other words, the balloon 3 may have the lower uterine lumen compression portion and the upper uterine lumen compression portion which compress each of the inner surfaces of the lower uterine lumen M1 and the upper uterine lumen M2. In this case, the drain flow path 21 and the supply-discharge flow path 22 may be provided one by one. Accordingly, the outer diameter of the tube 2 can be reduced, and the insertion resistance into the uterus can be reduced. In addition, the balloon 3 may be configured to include three or more balloons.

In the above-described embodiment, the stylet 5 is formed of stainless steel, polypropylene or the like, in addition to pure aluminum or an aluminum alloy. However, the configuration is not limited thereto. That is, any composition of the stylet 5 may be adopted as long as the shape of the tube 2 (second tube 2B) can be maintained and the stylet 5 deforms when a strong force is applied thereto.

In the above-described embodiment, the stylet 5 is accommodated in the drain terminal 41, but the present invention is not limited to this. For example, a hole into which the stylet 5 can be inserted and removed may be formed in the second tube 2B so that the stylet 5 can be accommodated in the hole. Furthermore, if the first tube 2A and the second tube 2B have a certain degree of rigidity, the stylet 5 may not be provided.

In the above-described embodiment, the supply-discharge terminals 43A and 43B are connected to the two-way cock. However, the configuration is not limited thereto. For example, a one-way valve or a high-speed injection valve may be connected to the supply-discharge terminals 43A and 43B. Out of these, the one-way valve does not need to turn off a cock when a syringe is replaced during liquid supply. When the syringe is detached, the valve is opened to prevent a backflow from the respective balloons 3A and 3B sides. The high-speed injection valve is a valve in which two one-way valves are combined with each other. An infusion solution bag of distilled water or physiological saline solution is connected to an upper connection port, and the respective supply-discharge terminals 43A and 43B and the syringe are connected to each other. When the syringe is pulled, the valve on the infusion solution bag side is opened. The respective supply-discharge terminals 43A and 43B sides are closed, and the liquid flows into the syringe. On the other hand, when the syringe is pushed, the valve on the infusion solution bag side is closed. The valves on the respective supply-discharge terminals 43A and 43B sides are opened, and the liquid flows into the respective balloons 3A and 3B. Therefore, the liquid can be repeatedly injected into the respective balloons 3A and 3B without detaching the valve or the syringe.

### INDUSTRIAL APPLICABILITY

The balloon unit for uterine hemostasis is used after delivery, and can reliably suppress or stop the uterine bleeding.

### REFERENCE SIGNS LIST

1 balloon unit for uterine hemostasis
2 tube
2A first tube
2B second tube
3 balloon
3A first balloon (lower uterine lumen compression portion)
3B second balloon (upper uterine lumen compression portion)
4 connector
5 stylet
6 cap
21 drain flow path
21A first drain flow path
21B second drain flow path
22 supply-discharge flow path
22A first supply-discharge flow path
22B second supply-discharge flow path
23 detection member
23A first detection member
23B second detection member
41 drain terminal
42A first supply-discharge tube
42B second supply-discharge tube
43 supply-discharge terminal
43A first supply-discharge terminal
43B second supply-discharge terminal
211A, 211B opening
222A, 222B opening
411 opening
Ar1 accommodation portion
M mother's body
M1 lower uterine lumen
M2 upper uterine lumen

1. A balloon unit for uterine hemostasis comprising:
a flexible tube; and
a balloon provided in a distal end portion of the tube,
wherein the balloon has a lower uterine lumen compression portion that compresses an inner surface of a lower uterine lumen and an upper uterine lumen compression portion that compresses an inner surface of an upper uterine lumen.

2. The balloon unit for uterine hemostasis according to claim 1,
wherein as the balloon, two balloons including a first balloon forming the lower uterine lumen compression portion and a second balloon forming the upper uterine lumen compression portion are provided.

3. The balloon unit for uterine hemostasis according to claim 2,
wherein maximum capacity of the first balloon is larger than maximum capacity of the second balloon.

4. The balloon unit for uterine hemostasis according to claim 3,
wherein the maximum capacity of the first balloon is 450 ml to 880 ml, the maximum capacity of the second balloon is 250 ml to 400 ml, and a ratio between the maximum capacity of the first balloon and the maximum capacity of the second balloon is 1.8 to 2.2:1.

5. The balloon unit for uterine hemostasis according to claim 2,
wherein the second balloon in an inflated state is disposed on a distal end side from the first balloon.

6. The balloon unit for uterine hemostasis according to claim 2,
wherein the tube has a first tube in which the first balloon is disposed, and a second tube in which the second balloon is disposed and which is inserted into the first tube.

7. The balloon unit for uterine hemostasis according to claim 6,
wherein the second balloon in a deflated state is accommodated inside the first tube.
